(19) United States
(12) Patent Application Publication
(10) Pub. No.: US 2021/0370001 A1
(43) Pub. Date: Dec. 2, 2021

(54) MASK DEVICE, MASK AND EXHALATION DETECTION METHOD

(71) Applicant: ATOM MEDICAL CORPORATION, Bunkyo-ku, Tokyo (JP)

(72) Inventors: Keisuke WAKABAYASHI, Saitama City, Saitama (JP); Shinichi KOBAYASHI, Saitama City, Saitama (JP); Takashi TANABE, Saitama City, Saitama (JP); Ichiro MATSUBARA, Bunkyo-ku, Tokyo (JP); Kazuya NIWA, Nagoya-shi, Aichi (JP)

(21) Appl. No.: 17/286,108

(22) PCT Filed: Oct. 15, 2019
(86) PCT No.: PCT/JP2019/040486

§ 371 Date: Apr. 16, 2021

(30) Foreign Application Priority Data
Oct. 18, 2018 (JP) 2018-196386

(51) Int. Cl. A61M 16/06; A61M 16/00; A61M 16/08

(52) U.S. Cl. CPC A61M 16/06; A61M 16/0003; A61M 2202/0208; A61M 2016/0042; A61M 16/0875

(57) ABSTRACT

A mask device which can appropriately detect exhalation of a patient is provided with a mask body installed on the patient, an exhalation tube having an exhalation flow path through which the exhalation of the patient runs and used for detecting the exhalation, and an oxygen tube used for supplying oxygen to the patient; the mask body is provided with an oxygen supply port to which the oxygen tube is connected and an exhalation tube support part which can support the exhalation tube; and the exhalation tube support part is structured so as to support the exhalation tube with changing at least one distance of a distance between a tip end of the exhalation tube and a nose part of the patient or a distance between the tip end of the exhalation tube and the mouth part of the patient.

### BACKGROUND OF THE INVENTION

### Technical Field

The present invention relates to a mask device, a mask and an exhalation detection method which can detect breathing of a patient. Priority is claimed on Japanese Patent Application No. 2018-196386, filed Oct. 18, 2018, the content of which is incorporated herein by reference.

### Background Art

Conventionally, an oxygen-supply mask for a patient for supplying oxygen (oxygen gas) to the patient is known (e.g., refer to Patent Document 1). A tube is connected to a connection part formed in a center part of this mask. This tube is provided with an oxygen-supply path for supplying oxygen into the mask, and a breath-ventilation path for discharging the breath of the patient to an exterior of the mask provided inside the oxygen-supply path. Oxygen is supplied from the oxygen-supply path into the mask; and the breath (carbon dioxide) of the patient is discharged from the breath-ventilation path to the exterior of the mask. The breath discharged from the breath-ventilation path to the exterior of the mask is supplied to an exhalation detector; and the breath of the patient is detected.

### CITATION LIST

[Patent Document 1] U.S. Patent Application Publication No. 2006/0196510 Specification

### SUMMARY OF INVENTION

### Technical Problem

Normally, the center part of the mask is fixed on a vicinity of a nose of the patient; however, since the tube having the breath-ventilation path is connected to the center part of the mask in the structure of Patent Document 1, it is difficult to ventilate the breath of the patient through the breath-ventilation path appropriately, for example, in a case in which the breath of the patient is weak exceedingly. When it is not possible to ventilate the breath of the patient through the breath-ventilation path, it is difficult to detect the breath of the patient for the exhalation detection device.

The present invention is achieved in consideration of the above circumstances, and has an object to provide a mask device, a mask, and an exhalation detection method which can detect the breath of the patient appropriately.

### Solution to Problem

A mask device of the present invention is provided with a mask body installed so as to cover a nose part and a mouth part of a patient; an exhalation tube having an exhalation flow path through which breath of the patient flows and used for detecting the breath; and an oxygen tube used for supplying oxygen to the patient. In the mask device, the mask body is provided with an oxygen supply port to which the oxygen tube is connected and an exhalation tube support part which can support the exhalation tube; and the exhalation tube support part is structured to support the exhalation tube with changing at least one distance of a distance between a tip end of the exhalation tube and the nose part or a distance between the tip end of the exhalation tube and the mouth part when the mask body is installed on the patient.

In the present invention, since the above-described exhalation tube support part is provided, the exhalation tube can be supported with changing at least one of the distances between the tip end of the exhalation tube and the nose part of the patient or the tip end of the exhalation tube and the mouth part of the patient. Accordingly, the breath of the patient can appropriately flow through the exhalation tube, and the breath of the patient can be appropriately detected by the mask device of the present invention. According to the present invention, since the above-described exhalation tube support part is provided, the tip end of the exhalation tube can be easily fixed on the nose part or the mouth part according to the breathing state (nasal respiration or oral respiration) of the patient without fixing means such as a tape or the like.

As a desirable aspect of the mask device of the present invention, it is preferable that the exhalation tube support part have a projecting support portion which projects from the mask body; the projecting support portion be structured to support the exhalation tube in a state in which the tip end of the exhalation tube turns toward a vicinity of the nose part; and be also structured to support the exhalation tube in a state in which the tip end of the exhalation tube turns toward a vicinity of the mouth part.

In the above-described aspect, since it can be disposed in a state in which the tip end of the exhalation tube turns toward either the nose part or the mouth part of the patient, the tip end of the exhalation tube can be fixed on an appropriate position in accordance with the breath (nasal respiration or oral respiration) of the patient.

As a desirable aspect of the mask device of the present invention, it is preferable that the projecting support portion be a supporting member which projects from at least a right side or a left side of the oxygen supply port on an inner surface of the mask body and supports the exhalation tube slidably along the inner surface of the mask body.

In the above-described aspect, since the exhalation tube can be slid along the inner surface of the mask body, the tip end of the exhalation tube can be disposed in a vicinity of the nose part or a vicinity of the mouth part of the patient so that the tip end of the exhalation tube is optimum regarding the stream of the breath discharged from the nose part or the mouth part of the patient. Accordingly, the breath of the patient can be more certainly detected. Since the supporting member slidably supports the exhalation tube, the distance between the nose part or the mouth part and the tip end of the exhalation tube can be adjusted.

As a desired aspect of the mask device of the present invention, it is preferable that on the mask body, at least one open hole be formed to ventilate between an outside and an inside of the mask body; the exhalation tube support part be notched at a part of an inner peripheral edge of the open hole and have a notch-like support portion formed of a notch part to which the exhalation tube can be fit-inserted; and the notch-like support portion support the exhalation tube slidably along a longitudinal direction of the exhalation tube.

In the above-described aspect, since the exhalation tube can be moved in the longitudinal direction of the exhalation tube (an axis direction of the exhalation tube, a front-and-behind direction of the mask body) in a state in which the exhalation tube is fit-inserted in the notch-like support portion, the tip end of the exhalation tube can be disposed at a position in which the breath of the patient can be more easily detected. Accordingly, the breath of the patient can be more certainly detected.

As a desirable aspect of the mask device of the present invention, it is preferable that a left-and-right pair of open holes be formed as the open hole, and the notch-like support portion be provided at least one of the pair of the open holes.

As a desired aspect of the mask device of the present invention, it is preferable that a left-and-right pair of first open holes and a second open hole positioned at a lower side of the first open holes be formed as the open holes; and the notch-like support portion be provided at least one of the pair of the open holes and the second open hole.

As a desired aspect of the mask device of the present invention, it is preferable that the exhalation tube support part have connector-connection parts provided on a surface of the mask body and connected to the exhalation tubes, and communication holes be formed on the respective connector-connection parts to communicate an outside and an inside of the mask body.

In the above-described aspect, by connecting the exhalation tube to any of the connector-connection parts, the distance between the tip end of the exhalation tube and the nose part or the mouth part of the patient can be easily changed. For instance, when one connector-connection part is positioned in the vicinity of the nose part when the mask body is installed and the other connector-connection parts are positioned in the vicinity of the mouth part, by connecting the exhalation tube to the appropriate connector-connection part in accordance with the breath condition of the patient, the breath of the patient can be certainly detected.

A mask of the present invention includes a mask body for being installed to cover a nose part and a mouth part of a patient. In the mask, the mask body is provided with an oxygen tube connection part to which an oxygen tube used for supplying oxygen to the patient is connected and an exhalation tube support part which supports an exhalation tube used for detecting breath of the patient; and the exhalation tube support part is structured so as to support the exhalation tube with changing at least one distance of a distance between a tip end of the exhalation tube and the nose part and a distance between the tip end of the exhalation tube and the mouth part when the mask body is installed on the patient.

An exhalation detection method of the present invention is an exhalation detection method detecting exhalation of a patient using a mask device comprising a mask body for being installed to cover a nose part and a mouth part of the patient, an exhalation tube having an exhalation flow path to let the exhalation of the patient flow through, and an oxygen tube used for supplying oxygen to the patient. In the exhalation detection method, a mask body provided with an oxygen supply port to which the oxygen tube is connected and an exhalation tube support part which can support the exhalation tube is used as the mask body; the exhalation tube support part is structured so as to support the exhalation tube with changing at least one distance of distances between a tip end of the exhalation tube and the nose part or the tip end of the exhalation tube and the mouth part when the mask body is installed on the patient; and the exhalation of the patient is detected with changing the distance between the tip end of the exhalation tube supported on the exhalation tube support part and the nose part or the mouth part.

In the present invention, by using the above-described mask device, at least one distance between the tip end of the exhalation tube and the nose part of the patient or between the tip end of the exhalation tube and the mouth part of the patient can be changed, so that the exhalation of the patient can appropriately flow through the exhalation tube, and the exhalation of the patient can be appropriately detected.

As a desired aspect of the exhalation detection method of the present invention, it is preferable that the exhalation tube support part has a projecting support portion projecting from the mask body; and the exhalation of the patient is detected by turning the tip end of the exhalation tube toward a vicinity of the nose part or a vicinity of the mouth part in a state in which the exhalation tube is supported on the projecting support portion.

In the above-described aspect, since the exhalation tube can be disposed in either state in which the tip end turns toward the nose part or the mouth part of the patient, the tip end of the exhalation tube can be fixed on an appropriate position in accordance with the respiration of the patient (nasal respiration or oral respiration).

As a desired aspect of the exhalation detection method of the present invention, it is preferable that at least one open hole is formed on the mask body to ventilate the mask body between an outside and an inside, and the exhalation tube is supported on the projecting support portion through the open hole.

In the above-described aspect, the exhalation tube can be supported on the projecting support portion through the open hole without arranging the exhalation tube on a contact surface of an edge portion (a flange part) of the mask body and skin of the patient.

Thereby preventing problems (e.g., discomfort, skin problems and the like of the patient)) resulting from that the exhalation tube is in contact with the skin for a long time before it happens.

Advantageous Effects of Invention

According to the present invention, the exhalation of the patient can be appropriately detected.

### BRIEF DESCRIPTION OF DRAWINGS

FIG. 1 is a schematic view showing an exhalation detection device including a mask device according to an embodiment of the present invention.

FIG. 2 is a frontal view of a mask body forming an exhalation detection device in the above-described embodiment.

FIG. 3 is a view showing the mask body in the above-described embodiment with enlarging a connector connection part of the mask body and a connector of an exhalation tube.

FIG. 4 is a perspective view showing a state in which the exhalation tube is connected to the connector connection part of the mask body in the above-described embodiment.

FIG. 5 is a perspective view showing a state in which the exhalation tube is installed on a notch part of the mask body in the above-described embodiment.

FIG. 6 is a rear view showing a state in which the exhalation tube is installed on a supporting member at a right side of the mask body in the above-described embodiment.

FIG. 7 is a rear view showing a state in which the exhalation tube is installed on the supporting member at a left side of the mask body in the above-described embodiment.

FIG. 8 is a rear view showing a state in which the exhalation tube is installed on the supporting member at the right side of the mask body in the above-described embodiment.

FIG. 9 is a cross-sectional view of the supporting member cut along the line A1-A1 shown in FIG. 6.

FIG. 10 is a frontal view showing a state in which the mask device in the above-described embodiment.

FIG. 11 is a frontal view of the mask body according to a modified example of the above-described embodiment.

### DESCRIPTION OF EMBODIMENTS

Below, an embodiment of a mask device, a mask, and an exhalation detection method of the present invention will be explained with referring drawings.

### [Schematic Structure of Exhalation Detection Device]

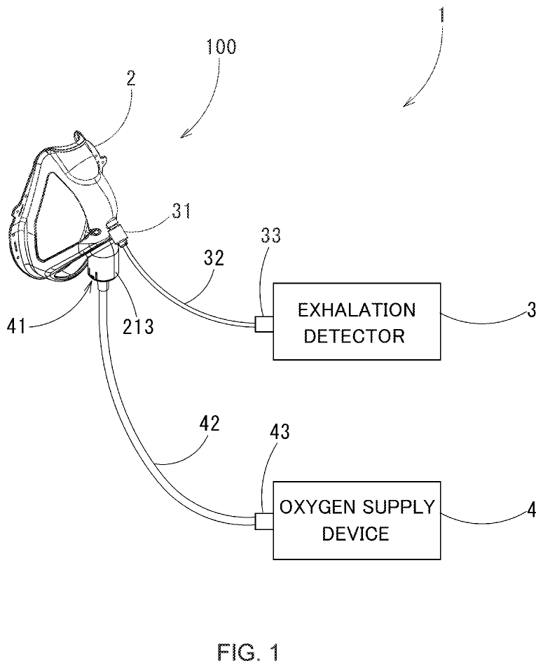

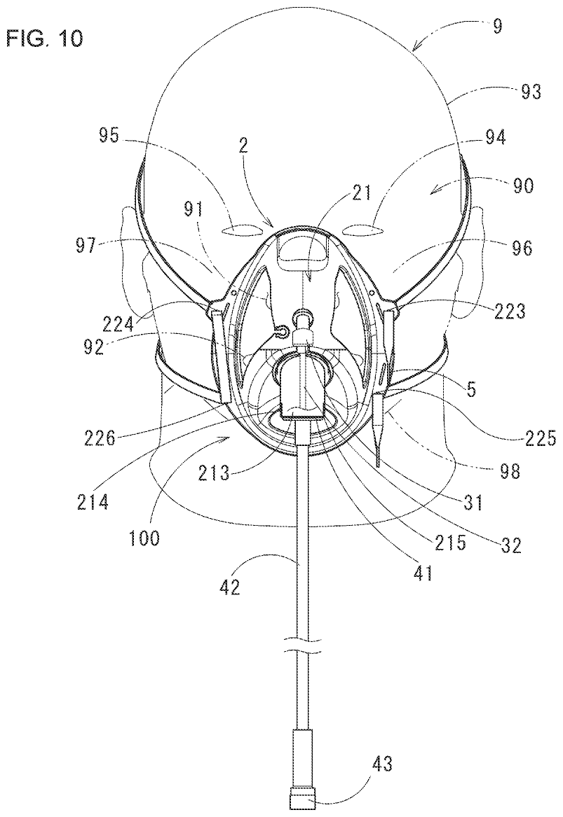

An exhalation detection device 1 of the present invention is provided with a mask device 100 (a mask device of the present invention), an exhalation detector 3 obtaining breathing information by detecting breath of a patient 9 (refer to FIG. 10) flowing from the mask device 100, and an oxygen supply device 4 supplying oxygen (oxygen gas) to the mask device 100, as shown in FIG. 1. As shown in FIGS. 1 and 10, the mask device 100 is provided with a mask (a mask of the present invention) having a mask body 2 put so as to cover a nose and a mouth of the patient 9, an exhalation tube 32 having an exhalation flow path flowing the breath of the patient 9 and connectable to the exhalation detector 3, and an oxygen tube 42 used for supplying oxygen.

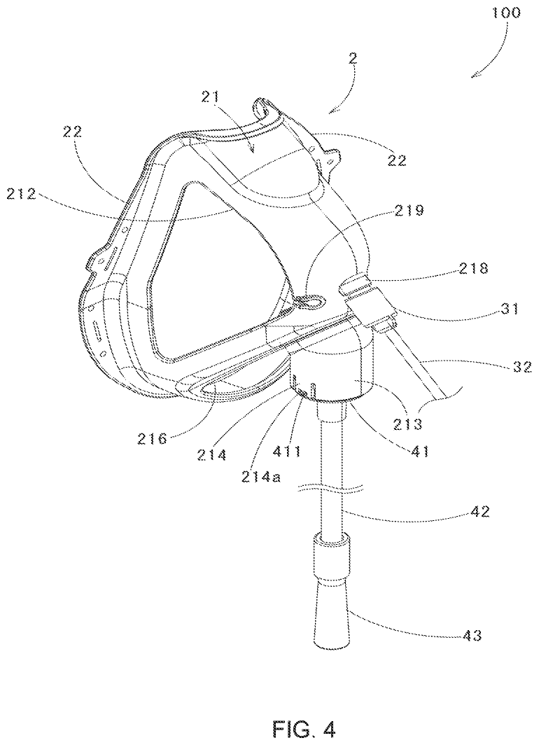

A connector 31 is fixed to a tip end of the exhalation tube 32; and an external connection connector 33 is fixed to a base end of the exhalation tube 32. The external connection connector 33 is connected to a connecter connection part (not shown) provided on the exhalation detector 3. For example, as shown in FIG. 4, by connecting the connector 31 to a connector-connection part 218 (a connector-connection part of the present invention) formed on an exterior surface of the mask body 2, the breath of the patient 9 is supplied to the exhalation detector 3 via the exhalation tube 32 and the external connection connector 33.

In the present embodiment, the exhalation detector 3 is structured by a carbon dioxide concentration detector, and has functions of detecting the carbon dioxide concentration from the breath of the patient and obtaining breathing information such as respiratory rate of the patient and the like.

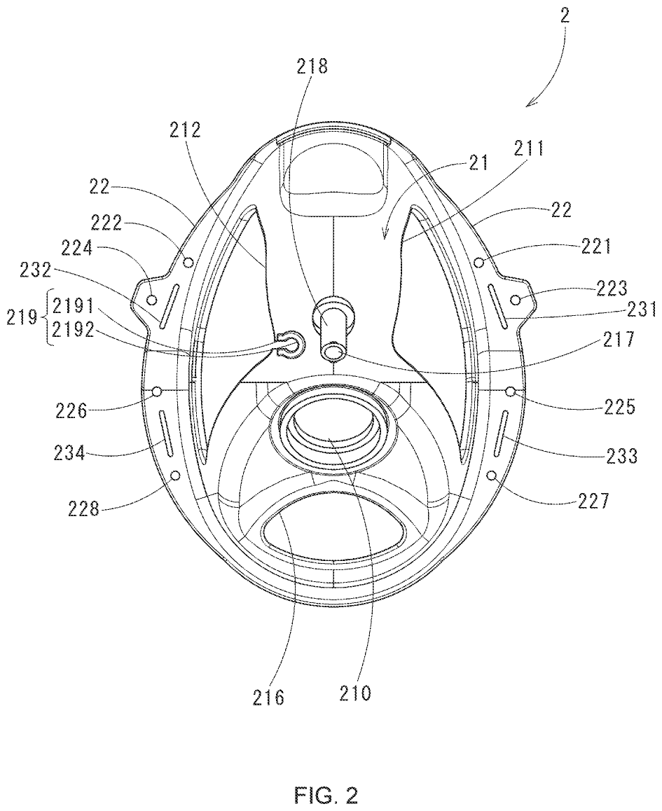

The oxygen tube 42 is fixed to a tube connector 41 at a distal end thereof and also fixed to an external connection connector 43 at a base end thereof. The external connection connector 43 is connected to a connector-connection part (not shown) provided at the oxygen supply device 4. The tube connector 41 is connected to a connector install part 213 (an oxygen tube connection part) disposed on an oxygen supply port 210 (refer to FIG. 2) of the mask body 2. The exhalation-detection device 1 having such a structure detects the breathing of the patient and obtains the breathing information, with supplying oxygen to the patient 9.

### [Structure of Mask Body]

The mask body 2 is installed on to cover a nose part 91 and a mouth part 92 of the patient as shown in FIG. 10. An installing cord 5 is attached to an outer peripheral part of the mask body 2 and possible to be hanged on a head part 93 of the patient 9. The mask body 2 is, for example, formed from mostly transparent (in other words, translucent) soft synthetic resin such as soft vinyl chloride or the like into a symmetrical shape with the left and the right.

In FIG. 10, the numeral symbol 94 indicates a left eye of the patient 9; and the numeral symbol 95 denotes a right eye of the patient 9. Hereinafter, in the present embodiment, the direction of left and right of the mask body 2 is explained where the side of the right eye 95 of the patient 9 on which the mask body 2 is put is the right direction (the right side) and the side of the left eye 94 of the patient 9 is the left direction (the left side). That is to say, in FIG. 2, the left side of the drawing is the right side of the mask body 2 and the right side of the drawing is the left side of the mask body 2.

In the mask body 2, as shown in FIG. 1, FIG. 2 and FIGS. 4 to 8, a dome-shaped cover part 21 is formed on a center part so as to form space between the nose part 91 and the mouth part 92 of the patient 9; and flange parts 22 abutting on a face 90 of the patient 9 is provided at an outer peripheral part of the cover part 21. On the flange parts 22, round cord-attaching holes 221 to 228 to which the installing cord 5 is put are formed four to the left and the right. For example, as shown in FIG. 10, four cord-attaching holes are selected from the cord-attaching holes 221 to 228, and it is attached to the patient 9 using the installing cord 5. On the left and right flange parts 22, two pairs of bandage-attaching slots 231 to 234 through which a bandage (not shown) can put are formed, which are different from the cord-attaching holes 221 to 228 (refer to FIG. 2).

Respectively on both left and right sides of the cover part 21 formed in a dome shape, left-and-right pair of ventilation open holes 211 and 212 (open holes or first holes of the present invention) having substantially a triangular shape are formed at an upper side for ventilating the cover part 21 between the interior and the exterior. The ventilation open holes 211 and 212 are disposed at the position corresponding to the left and right nostrils and the vicinity of the nostrils of the patient 9 in a state in which the mask body 2 is mounted on the patient 9.

Specifically, the ventilation open holes 211 and 212 are formed to have a size through which the exhalation tube 32 can pass as shown in FIG. 2. The longest line among three lines forming the substantially triangle ventilation open holes 211 and 212 extends along the flange parts 22 from the upper side toward the lower side in a gentle curve-line shape; from both ends of the longest line, the other two lines extend toward a center of the mask body 2; these two lines intersect at a shifted position to a side from a position between the connector install part 213 and the connector-connection part 218. That is to say, one of vertexes of the ventilation open holes 211 and 212 having substantially triangular shape is positioned in the vicinity of the center of the mask body 2. At the center position in the left-and-right direction below the ventilation open holes 211 and 212 (the position corresponding to the periphery of the mouth part 92 in a state in which the mask body 2 is attached to the patient 9), a second ventilation open hole 216 being substantially an oval shape (an open hole or a second open hole of the present invention) is formed.

The oxygen supply port 210 is formed at the position slightly below the center position of the cover part 21; on the oxygen supply port 210, the connector install part 213 being cylindrical shape having a communication hole communicating with the oxygen supply port is disposed (refer to FIG. 4). The tube connector 41 of the oxygen tube 42 is attached to the connector install part 213. The connector install part 213 is formed so that a tip end of the cylinder toward the lower side of the mask body 2. On the tip end of the cylinder of the connector install part 213, two notched-piece parts 214 and 215 (refer to FIG. 4 and FIG. 10) are formed. On the notched-piece parts 214 and 215, a locking hole 214a is formed to prevent the tube connector 41 from falling down by locking claw part 411 of the tube connector 41 when the tube connector 41 is attached to the connector install part 213.

The tube connector 41 is formed from hard synthetic resin such as substantially transparent hard vinyl chloride or the like, for example. Similarly, the oxygen tube 42 is formed from soft synthetic resin such as substantially transparent soft vinyl chloride or the like, for example; the tube connector 41 is attached to one end of the oxygen tube 42, and it is attached to the mask body 2 via the tube connector 41. To the other end of the oxygen tube 42, a gas cylinder such as an oxygen tank provided on the oxygen supply device 4, the external connection connector 43 which is connected to a nozzle for supplying gas and the like provided in a hospital room or the like are attached. Accordingly, an oxygen gas flow is taken into the space inside the mask body 2 via the connector install part 213.

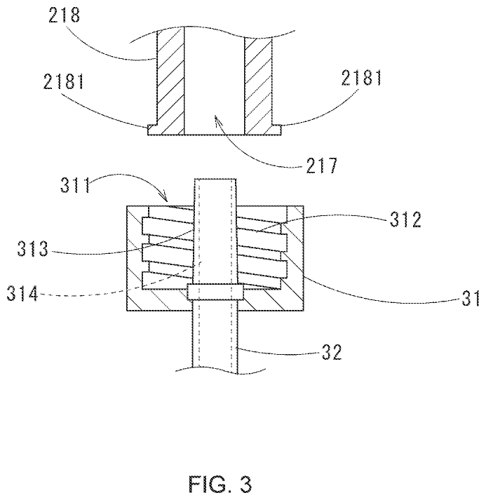

The connector 31 is formed from hard synthetic resin such as hard vinyl chloride, soft synthetic resin, or the like, for example. The connector 31 is what is called a Luer lock connector having an open mouth part 311 and a thread groove 312 formed on a peripheral surface of the open mouth part 311, as shown in FIG. 3. In the open mouth part 311, a protrusion 313 toward the opening end is formed; on the protrusion 313 in which a communication hole 314 is formed for inserting the breath into the exhalation tube 32.

The exhalation tube 32 is formed from soft synthetic resin having elasticity such as substantially transparent soft vinyl chloride for example; the connector 31 is attached to the tip end of the exhalation tube 32. The base end of the exhalation tube 32 is connected to the external connection connector 33; by connecting the external connection connector 33 to the exhalation detector 3, the breath of the patient is supplied to the exhalation detector 3. The external connection connector 33 is formed from hard synthetic resin such as hard vinyl chloride or the like for example, similarly to the connector 31.

### <Structure of Support Part of Exhalation Tube>

### [Structure of Connector-Connection Part]

A connector-connection part 218 (a connector-connection part of the present invention) in which a connection hole 217 shown in FIG. 2 and FIG. 3 is formed is fixed on the position slightly above the center position of the cover part 21 of the mask body 2. The connector-connection part 218 is formed from hard synthetic resin such as hard vinyl chloride, for example. The connector-connection part 218 is a cylindrical portion in which the cover part 217 for communicating the front side and the inside of the mask body 2 (the cover part 21) is formed. On the outer peripheral part of the tip end of the connector-connection part 218, a pair of projections 2181 is formed so as to protrude in the opposite direction of 180° is formed. The pair of the projections 2181 is threaded to the thread groove 312 of the connector 31. Accordingly, as shown in FIG. 1 and FIG. 4, when the connector 31 is connected to the connector-connection part 218, the protrusion 313 of the connector 31 is inserted into the cover part 217 of the connector-connection part 218 and the connector 31 is rotated in one direction (e.g., the right direction), so that the pair of the projections 2181 is threaded to the thread groove 312 of the connector 31 and the connector 31 is rigidly fixed to the connector-connection part 218.

Since the connector-connection part 218 is formed at the position slightly above the center position of the mask body 2, the connector-connection part 218 is positioned in the vicinity of the nose part 91 when the mask body 2 is mounted on the patient 9. Thereby, the exhalation of the patient 9 discharged from the nose part 91 is supplied to the exhalation detector 3 via the exhalation tube 32. That is to say, the connector-connection part 218 is one of a plurality of connector-connection parts constituting the exhalation tube support part of the present invention, supporting the exhalation tube 32 to position the tip end of the exhalation tube 32 in the vicinity of the nose part 91 of the patient 9.

### [Structure of Notch Part]

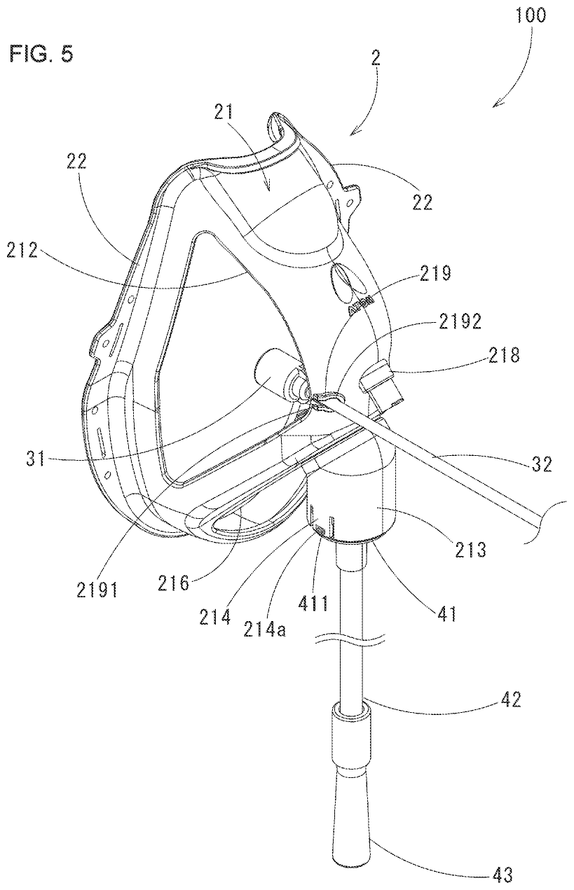

As shown in FIG. 2, FIG. 4, and FIG. 5, on a periphery of the ventilation open hole 212 of the cover part 21, a notch part 219 (a notch-like support portion of the present invention) is formed supporting the exhalation tube 32 slidably and fittably along the length direction of the exhalation tube 32 (an axis direction of the exhalation tube 32, a front-and-behind direction of the mask body 2). The notch part 219 is provided with an insertion part 2191 extending from a top of substantially the triangle ventilation open hole 212 toward the center of the cover part 21 and a fit-insertion part 2192 positioned at the center side of the cover part 21 than the insertion part 2191 and having a larger diameter than an open width of the insertion part 2191. The open width of the insertion part 2191 is set to a width smaller than a diameter of the exhalation tube 32 and enabling the exhalation tube 32 to pass through the fit-insertion part 2192 via the insertion part 2191. The fit-insertion part 2192 is formed to be a round shape with a diameter substantially the same as or slightly smaller than the diameter of the exhalation tube 32, for example. The notch part 219 is a structure supporting the exhalation tube 32, so that the periphery part (a part of the insertion part 2191 and the fit-insertion part 2192) is formed to be thicker than the cover part 21.

In a state in which the tip end of the exhalation tube 32 is disposed at the inner surface side of the mask body 2 regarding the insertion part 2191 and the base end side of the exhalation tube 32 is disposed at the outer surface side of the mask body 2, that is, in a state in which the exhalation tube 32 passes through the ventilation open holes 212 and the tip end of the exhalation tube 32 is disposed inside the mask body 2, the exhalation tube 32 can be attached to the fit-insertion part 2192 by sliding the exhalation tube 32 along the insertion part 2191 toward the center side (the fit-insertion part 2192 side). Since the peripheral edge part of the fit-insertion part 2192 (the notch part 219) is formed thick, it is possible to reduce undesirable looseness after fitting the exhalation tube 32 to the fit-insertion part 2192.

In the present embodiment, the notch part 219 is formed on the peripheral part of the ventilation open holes 212 provided at the right side of the mask body 2; however, it is not limited to this, for example, it is may be formed on the peripheral part (the vicinity of the top at the center part side) of the ventilation open holes 211 formed on the left side of the mask body 2. That is, the notch part 219 may be formed on the either peripheral part of the ventilation open holes 211 and 212. The notch part 219 may be formed on both peripheral parts of the ventilation open holes 211 and the ventilation open holes 212; however, in order not to extremely lower the strength of the cover part 21 at the center part, it may be formed on the peripheral part of only one of the ventilation open holes 211 and 212 or formed on different parts (asymmetry parts in the left and right) on both peripheral parts of the ventilation open holes 211 and 212. Moreover, the notch part 219 may be formed on the peripheral part in the vicinity of the mouth part 92 of the second ventilation open hole 216.

### [Structure of Supporting Members]

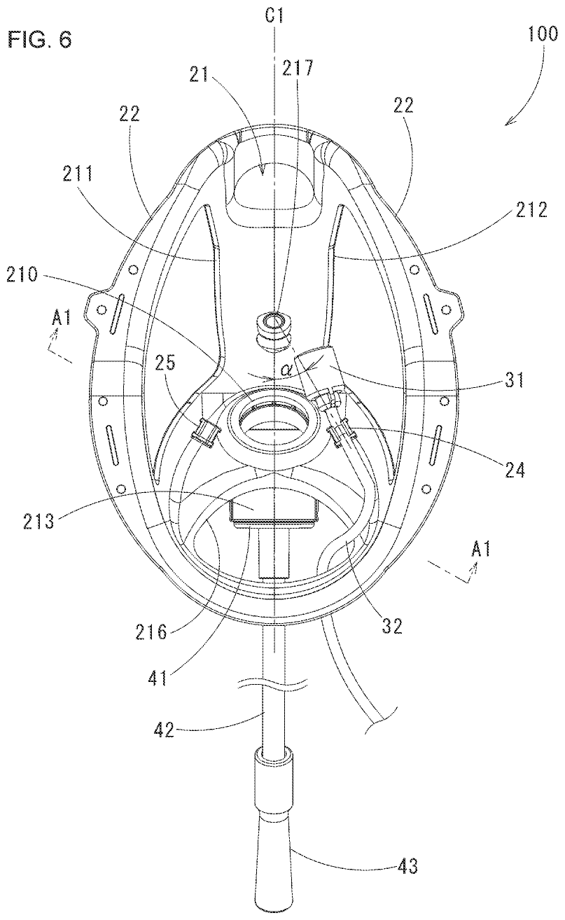

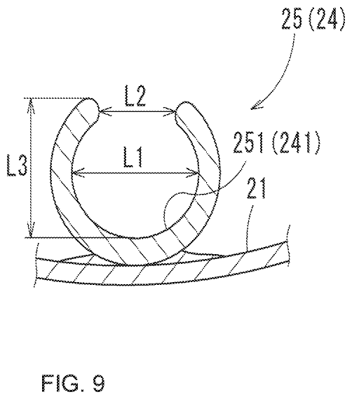

As shown in FIGS. 6 to 8, on the inner surface of the cover part 21 (an inner (the patient 9 side) surface when the patient 9 wears the mask body 2), a pair of left and right supporting members 24 and 25 (projecting support portions of the present invention) are provided which protrude from the inner surface of the cover part 21 and slidably support the mask body 23 along the inner surface of the mask body 2. The supporting members 24 and 25 are, for example, formed from hard synthetic resin such as hard vinyl chloride. The supporting members 24 and 25 are welded respectively to the right side and the left side of the cover part 21 on the inner surface of the cover part 21, as shown in FIG. 9.

Specifically, the supporting member 24 is positioned between the ventilation open holes 212 and the oxygen supply port 210 in the right side of the cover part 21; and the supporting member 25 is positioned between the ventilation open holes 211 and the oxygen supply port 210 in the left side of the cover part 21. That is, the supporting member 24 is positioned on the vicinity of the right end part of the mouth part 92 when the mask body 2 is attached to the patient 9; and the supporting member 25 is positioned on the vicinity of the left end part of the mouth part 92 when the mask body 2 is attached to the patient 9.

The supporting members 24 and 25 are welded to the cover part 21; however, it is not limited to this, it may be bonded to the cover part 21 by adhesive agent or the like, or it may be formed integrally with the cover part 21 and the like.

The supporting members 24 and 25 have a form holding the exhalation tube 32 from both left and right sides, in other words, the supporting members 24 and 25 have a form to support by inserting the exhalation tube 32 into grooves 241 and 251 formed by the supporting members 24 and 25. A width L1 of the grooves 241 and 251 at substantially the center part (a maximum length corresponding to a diameter direction of the exhalation tube 32 when the exhalation tube 32 is inserted into the supporting members 24 and 251) is set to substantially the same as a diameter of the exhalation tube 32; a width L2 at an open end of the grooves 241 and 251 is set slightly smaller than the diameter of the exhalation tube 32; and a height dimension L3 of the grooves 241 and 251 is set to substantially the same as or slightly smaller than the diameter of the exhalation tube 32. A length of the supporting members 24 and 25 (a length along a longitudinal direction of the exhalation tube 32 when the exhalation tube 32 is inserted into the grooves 241 and 251) is set to more than a prescribed length (e.g., 3 mm or more). Accordingly, by installing the exhalation tube 32 to the supporting members 24 and 25, the exhalation tube 32 is held by the supporting members 24 and 25 with appropriate pressure.

The supporting members 24 and 25 support the tip end of the exhalation tube 32 slidably along substantially up and down direction of the mask body 2. Specifically, the supporting members 24 and 25 are respectively fixed so that groove parts 241 and 251 incline with a prescribed angle of “α” with respect to an imaginary line C1 extending from the upper end of the mask body 2 to the lower end through the center of the mask body 2 (refer to FIG. 6), and so that a distance between the supporting members 24 and 25 is increase from the upper to the lower. The angle “α” regarding the imaginary line C1 of the supporting members 24 and 25 is, for example, set to 5° to 45°, more preferably 25° to 35°. In the example shown in FIG. 6 to FIG. 8, the supporting members 24 and 25 are disposed to be line symmetry regarding the imaginary line C1.

The supporting members 24 and 25 are structured so as to rigidly support the exhalation tube 32 so that the angle of the exhalation tube 32 regarding the imaginary line C1 is 5° to 45°, more preferably 25° to 35° and slidably support the exhalation tube 32 along the length direction of the supporting members 24 and 25.

Here, in a case in which the supporting members 24 and 25 are disposed parallel to the imaginary line C1 (i.e., α=0°), since it is necessary to arrange the exhalation tube 32 outside from the vicinity of the center part of the second ventilation open hole 216 if the connector 31 is intended to be disposed so as to detect the exhalation from the nose part, the exhalation tube 32 is easily obstructive when the patient drinks the beverage. In a case in which the supporting members 24 and 25 are disposed perpendicular to the imaginary line C1 (i.e., α=90°), the exhalation from the nose part may be not easily detected because the tip end of the connector 31 is lateral and it may not be easy to dispose the exhalation tube 32 (the connector 31) so as not to be obstructive with the position of the oxygen supply port 210.

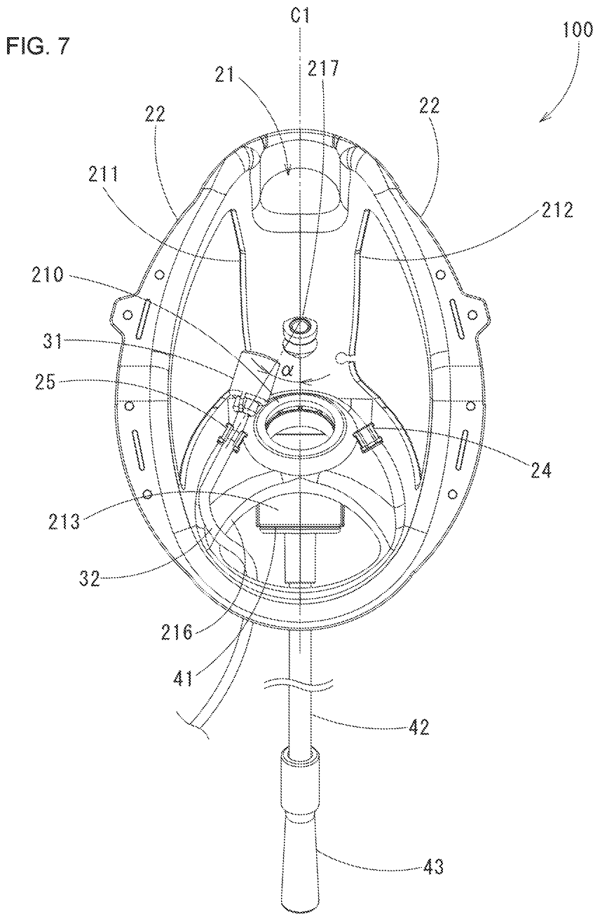

On the other hand, in the present invention, since the degree “α” regarding the imaginary line C1 of the supporting members 24 and 25 is set in the above-mentioned range, as shown in FIG. 7, the exhalation tube 32 can be extended between the ventilation open holes 211 and the second ventilation open hole 216 and the exhalation tube 32 can be arranged outside the mask body 2 from the side of the second ventilation open hole 216. Accordingly, the exhalation tube 32 is not easily obstructive to drink the beverage. The exhalation tube 32 (the connector 31) can be disposed using the space between the ventilation open holes 211 and the second ventilation open hole 216, so that the exhalation from the nose part can be appropriately detected and the exhalation tube 32 (the connector 31) can be disposed without obstructing the position of the oxygen supply port 210.

Even in a case in which the exhalation tube 32 is connected to the supporting members 24 and 25 via any one of the ventilation open holes 211 and 212 or the second ventilation open hole 216, the exhalation tube 32 can be fixed without closing the ventilation open holes 211, 212, and 216. Especially, in a case in which the exhalation tube 32 passes through the second ventilation open hole 216 and fixed, the exhalation tube 32 is not obstructive even though the exhalation tube 32 is left being installed when drinking through a straw and a treatment such as oral health care, and it is possible to save effort of reinstalling of the exhalation tube 32 and reduce a risk of forgetting the installation.

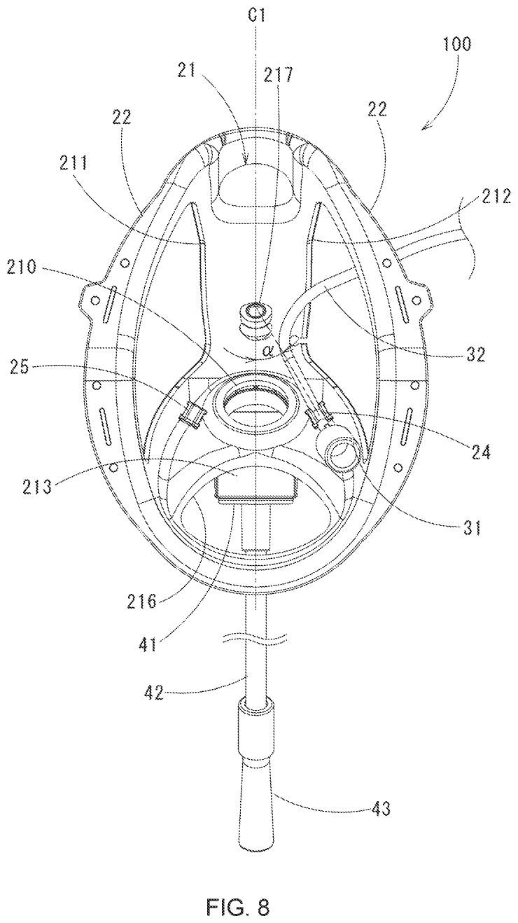

As shown in FIG. 6, when the exhalation tube 32 is attached to the supporting member 24 turning the tip end upward, the tip end of the exhalation tube 32 is arranged in the vicinity of the right nostril of the nose part 91 of the patient; and as shown in FIG. 7, when the exhalation tube 32 is attached to the supporting member 25 turning the tip end up ward, the tip end of the exhalation tube 32 is arranged in the vicinity of the left nostril of the nose part 91 of the patient. On the other, as shown in FIG. 8, when the exhalation tube 32 is attached to the supporting member 24 turning the tip end downward, the tip end of the exhalation tube 32 is arranged in the vicinity of the right end of the mouth part 92 of the patient; and when the exhalation tube 32 is attached to the supporting member 25 turning the tip end downward, the tip end of the exhalation tube 32 is arranged in the vicinity of the left end of the mouth part 92 of the patient.

In the present embodiment, the supporting members 24 and 25 support the exhalation tube 32 slidably along substantially a vertical direction of the mask body 2; accordingly, by installing the exhalation tube 32 on either one of the supporting members 24 and 25 turning the tip end to a prescribed direction and sliding it up and down, it is possible to move to a position where the exhalation of the patient can be more easily detected.

### [Method of Detecting Exhalation]

Next, a method of detecting exhalation using the above-described mask device 100 will be explained. First, doctors, nurses or the like (hereinafter, “doctors and the like”) confirm which the patient 9 breathes through the nose or the mouth. If the patient breathes through the nose, the exhalation tube 32 is selected to attach to either the exhalation tube 32, the connector-connection part 218, the notch part 219, or the supporting members 24 and 25. For example, when the exhalation tube 32 is connected to the connector-connection part 218, after the mask body 2 is attached on the head part 93 of the patient 9, and then the connector 31 is connected to the connector-connection part 218, so that the exhalation tube 32 is attached to the mask body 2. When the exhalation tube 32 is installed on the notch part 219, after the mask body 2 is attached on the head part 93 of the patient 9, the exhalation tube 32 is fit inserted to the fit-insertion part 2192 of the notch part 219 and the exhalation tube 32 is slid along the back-and-forth direction of the mask body 2 (the longitudinal direction of the exhalation tube 32), so that the tip end of the exhalation tube 32 is moved to a position where the exhalation can be most easily detected. That is, the tip end position of the exhalation tube 32 is adjusted by decreasing or increasing the distance between the nose part of the patient 9 and the tip end of the exhalation tube 32.

When the exhalation tube 32 is installed on the supporting members 24 and 25, for example, before the mask body 2 is installed on the head part 93 of the patient 9, as shown in FIG. 6 or FIG. 7, the exhalation tube 32 is installed on either one of the supporting members 24 and 25. Then the mask body 2 is installed on the head part 93 of the patient 9, and the exhalation tube 32 is slid along the inner surface of the mask body 2, so that the tip end of the exhalation tube 32 is moved to the position where the exhalation can be most easily detected.

When the exhalation tube 32 is installed on the supporting members 24 and 25, by installing the exhalation tube 32 on the supporting members 24 and 25 through the second ventilation open hole 216, clearance between the face 90 of the patient 9 and the flange parts 22 of the mask body 2 is not generated when the mask body 2 is installed on the head part 93. The exhalation tube 32 is installed on the supporting members 24 and 25 before the mask body 2 is installed on the head part 93 of the patient 9; however, it is not limited to this, the exhalation tube 32 may be installed on the supporting members 24 and 25 after the mask body 2 is installed on the head part 93 of the patient 9, a manner of installing is not specifically limited. The ventilation open hole through which the exhalation tube 32 passes is the second ventilation open hole 216; however, it is not limited to this, it may be the ventilation open holes 211 and 212.

On the other hand, in a case in which the doctors and the like confirms that the patient 9 breathes by the mouth, the doctors and the like choose that the exhalation tube 32 is installed on either one of the supporting members 24 and 25. When the exhalation tube 32 is installed on the supporting member 24, before the mask body 2 is installed on the head part 93 of the patient 9, as shown in FIG. 8, the exhalation tube 32 is installed on the supporting member 24 through the ventilation open holes 212 and the mask body 2 is installed on the head part 93 of the patient 9, and then the exhalation tube 32 is slid along the inner surface of the mask body 2, so that the tip end of the exhalation tube 32 is moved to a position where the exhalation can be most easily detected. When the exhalation tube 32 is installed on the supporting member 25, before the mask body 2 is installed on the head part 93 of the patient 9, the exhalation tube 32 is installed on the supporting member 25 through the ventilation open holes 211 and the mask body 2 is installed on the head part 93 of the patient 9, and then the exhalation tube 32 is slid along the inner surface of the mask body 2, so that the tip end of the exhalation tube 32 is moved to a position where the exhalation can be most easily detected.

As described above, by supporting the exhalation tube 32 by the connector-connection part 218 as a support part of the exhalation tube, the notch part 219 or the supporting members 24 and 25, the exhalation of the patient 9 is certainly flow through the exhalation flow path of the exhalation tube 32. When the exhalation is supplied to the exhalation detector 3 through the base end of the exhalation tube 32, the breathing information of the patient 9 can be obtained by the exhalation detector 3.

In the present embodiment, since it is structured to enable to change the distance between the tip end of the exhalation tube 32 and the nose part 91 of the patient 9 and the distance between the tip end of the exhalation tube 32 and the mouth part 92 of the patient 9, it is possible to easily fix the tip end of the exhalation tube in the vicinity of the nose part and mouth part of the patient. That is, only by change the exhalation tube support part (the supporting members 24 and 25, the notch part 219, and the connector-connection part 218) supporting the exhalation tube 32, the above-described distances can be changed. The tip end of the exhalation tube 32 can be supported in either state toward the vicinity of the nose part 91 or the mouth part 92 of the patient 9, so that the tip end of the exhalation tube 32 can be fixed on an appropriate position suitable for the breath (nasal respiration and oral respiration). Accordingly, the breath of the patient 9 can appropriately flow through the exhalation tube 32, so that the respiration of the patient can be appropriately detected by the mask device 100 of the present embodiment. Moreover, according to the present embodiment,

Since the supporting members 24 and 25 arranged at the right side and the left side of the oxygen supply port 210 can support the exhalation tube 32 along substantially the up-and-down direction of the mask body 2 (with slightly inclined at the degree “α”), in a case in which the exhalation tube 32 is installed on the supporting members 24 and 25 so that the tip end of the exhalation tube 32 turns upward, the tip end of the exhalation tube 32 can be arranged in the vicinity of the nostril of the nose part 91 of the patient 9; in a case in which the exhalation tube 32 is installed on the supporting members 24 and 25 so that the tip end of the exhalation tube 32 turns downward, the tip end of the exhalation tube 32 can be arranged in the vicinity of the mouth part 92 of the patient. Since the supporting members 24 and 25 slidably support the exhalation tube 32, the tip end of the exhalation tube 32 can be arranged in an appropriate position according to strength of the breath of the patient by adjusting the distances between the tip end of the exhalation tube 32 and the nose part 91 or the mouth part 92. Accordingly, the exhalation can be certainly detected even if the breath of the patient is weak.

Furthermore, since the exhalation tube 32 can be move along the longitudinal direction of the exhalation tube 32 in a state in which the exhalation tube 32 is put in the notch part 219, the tip end of the exhalation tube 32 can be arranged at an appropriate position according to the strength of the breath of the patient. That is, since the position of the tip end can be arranged at a position where the exhalation of the patient can be easily detected, the exhalation can be certainly detected even if the breath of the patient is weak.

In the above-described embodiment, since the exhalation tube 32 is installed on the supporting members 24 and 25 through the pair of the ventilation open holes 211 and 212 or the second ventilation open hole 216, it is reduced that the exhalation tube 32 is directly pushed against skin of the patient 9, and discomfort of the patient 9 and skin problems and the like can be prevented.

The present invention is not limited to the above-described embodiments and various modifications may be made without departing from the scope of the present invention. For example, the mask body 2 has the one connector-connection part 218 though, it is not limited to this, the two connector-connection parts 218 may be provided with the mask body 2 at different parts.

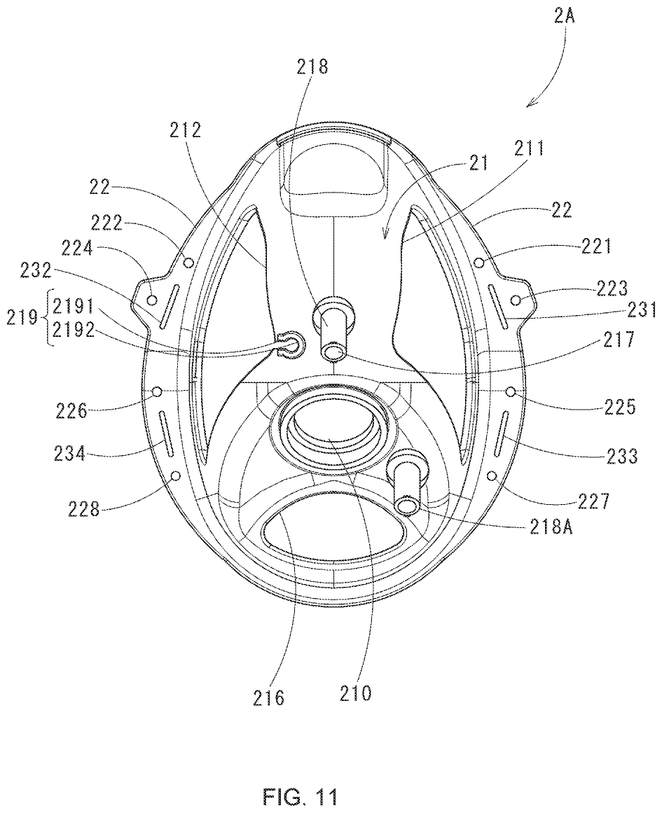

FIG. 11 is a frontal view of a mask body 2A in a modified example of the above-described embodiment. In this modified example, as shown in FIG. 11, a connector-connection part 218A is provided other than the connector-connection part 218 at slightly upper position than the center of the mask body 2A. The connector-connection part 218A is arranged at the right side of the oxygen supply port 210 of the mask body 2A, and arranged in the vicinity of the left edge part of the mouth part 92 of the patient 9 if the patient 9 wears the mask body 2. The connector-connection part 218A is formed in a same shape and from a same material as those of the connector-connection part 218, for example. Accordingly, in this modified example, the exhalation in the vicinity of the nose part 91 of the patient 9 can be detected if the connector 31 is connected to the connector-connection part 218; and the exhalation in the vicinity of the mouth part 92 of the patient 9 can be detected if the connector 31 is connected to the connector-connection part 218A. That is, in this modified example, only by changing the connecting object of the connector 31 of the exhalation tube 32, the distance between the tip end of the exhalation tube 32 and the nose part 91 and the mouth part 92 of the patient 9 can be easily changed, so that the exhalation of the patient 9 can be detected according to the breath condition of the patient 9.

The connector-connection parts 218 and 218A are provided in this modified example; however, it is not limited to this, more connector-connection part may be provided. The connector-connection part 218A is arranged at the right side of the oxygen supply port 210; however, it may be arranged at the left side of the oxygen supply port 210.

Two supporting members 24 and 25 are provided in the above embodiment; however, it is not limited to this, only one may be provided, for example. In the above embodiment, the supporting members 24 and 25 are the supporting members having a structure forming the grooves 241 and 251 respectively and holding the exhalation tube 32 by the grooves 241 and 251; however, it is not limited to this, it may be hooked shape, for example. That is, it does not matter for the shape if the exhalation tube 32 can be supported.

In the above embodiment, the connector-connection part 218, the notch part 219, and the supporting members 24 and 25 are provided as the exhalation tube support part; however, it is not limited to this, one or two among the connector-connection part, the notch part, and the supporting member may be provided. That is, the number and the type of the exhalation tube support part do not matter if at least one of the distances between the tip end of the exhalation tube 32 and the nose part or the mouth part of the patient can be changed.

### INDUSTRIAL APPLICABILITY

The present invention is installed on a patient and used for detecting an exhalation of the patient.

### REFERENCE SIGNS LIST

1 Exhalation detection device
2, 2A Mask body
3 Exhalation detector
4 Oxygen supply device
5 Installing cord
9 Patient
21 Cover part
210 Oxygen supply port
211, 212 Ventilation open hole
213 Connector install part
214, 215 Notched-piece part
216 Second ventilation open hole
217 Communication hole
218, 218A Connector-connection part (Exhalation tube support part)
219 Notch part
2191 Insertion part
2192 Fit-insertion part
22 Flange part
221 to 228 Cord-attaching hole
231 to 234 Bandage-attaching slot
24, 25 Supporting member
241, 251 Groove
31 Connector
32 Exhalation tube
33 External connection connector
41 Tube connector
42 Oxygen tube
43 External connection connector
90 Face
91 Nose part
92 Mouth part
93 Head part
94 Left eye
95 Right eye
100 Mask device

1. A mask device comprising:
a mask body for being installed so as to cover a nose part and a mouth part of a patient;
an exhalation tube having an exhalation flow path through which breath of the patient flows and used for detecting the breath; and
an oxygen tube used for supplying oxygen to the patient,
wherein the mask body is provided with an oxygen supply port to which the oxygen tube is connected and an exhalation tube support part which can support the exhalation tube, and
wherein the exhalation tube support part is structured to support the exhalation tube with changing at least one distance of a distance between a tip end of the exhalation tube and the nose part or a distance between the tip end of the exhalation tube and the mouth part when the mask body is installed on the patient.

2. The mask device according to claim 1, wherein the exhalation tube support part has a projecting support portion which projects from the mask body;
the projecting support portion is structured to support the exhalation tube in a state in which the tip end of the exhalation tube turns toward a vicinity of the nose part; and
is also structured to support the exhalation tube in a state in which the tip end of the exhalation tube turns toward a vicinity of the mouth part.

3. The mask device according to claim 2, wherein
the projecting support portion is a supporting member which projects from at least a right side or a left side of the oxygen supply port on an inner surface of the mask body and supports the exhalation tube slidably along the inner surface of the mask body.

4. The mask device according to claim 1, wherein
on the mask body, at least one open hole is formed to ventilate between an outside and an inside of the mask body;
the exhalation tube support part is notched at a part of an inner peripheral edge of the open hole and has a notch-like support portion formed of a notch part to which the exhalation tube can be fit-inserted; and
the notch-like support portion supports the exhalation tube slidably along a longitudinal direction of the exhalation tube.

5. The mask device according to claim 4, wherein
a left-and-right pair of open holes are formed as the open holes, and
the notch-like support portion is provided at least one of the pair of the open holes.

6. The mask device according to claim 4, wherein
a left-and-right pair of first open holes and a second open hole positioned at a lower side of the first open holes are formed as the open holes; and
the notch-like support portion is provided at least one of the pair of the open holes and the second open hole.

7. The mask device according to claim 1, wherein
the exhalation tube support part has connector-connection parts provided on a surface of the mask body and connected to the exhalation tubes, and
communication holes are formed on the respective connector-connection parts to communicate an outside and an inside of the mask body.

8. A mask comprising a mask body for being installed to cover a nose part and a mouth part of a patient, wherein
the mask body is provided with an oxygen tube connection part to which an oxygen tube used for supplying oxygen to the patient is connected and an exhalation tube support part which supports an exhalation tube used for detecting breath of the patient; and
the exhalation tube support part is structured so as to support the exhalation tube with changing at least one distance of a distance between a tip end of the exhalation tube and the nose part and a distance between the tip end of the exhalation tube and the mouth part when the mask body is installed on the patient.

9. An exhalation detection method detecting exhalation of a patient using a mask device comprising a mask body for being installed to cover a nose part and a mouth part of the patient, an exhalation tube having an exhalation flow path to let the exhalation of the patient flow through, and an oxygen tube used for supplying oxygen to the patient, wherein
a mask body provided with an oxygen supply port to which the oxygen tube is connected and an exhalation tube support part which can support the exhalation tube is used as the mask body;
the exhalation tube support part is structured so as to support the exhalation tube with changing at least one distance of distances between a tip end of the exhalation tube and the nose part or the tip end of the exhalation tube and the mouth part when the mask body is installed on the patient; and
the exhalation of the patient is detected with changing the distance between the tip end of the exhalation tube supported on the exhalation tube support part and the nose part or the mouth part.

10. The exhalation detection method according to claim 9, wherein
the exhalation tube support part has a projecting support portion projecting from the mask body; and
the exhalation of the patient is detected by turning the tip end of the exhalation tube toward a vicinity of the nose part or a vicinity of the mouth part in a state in which the exhalation tube is supported on the projecting support portion.

11. The exhalation detection method according to claim 10, wherein
at least one open hole is formed on the mask body to ventilate the mask body between an outside and an inside, and
the exhalation tube is supported on the projecting support portion through the open hole.
